(19) United States
(12) Patent Application Publication
(10) Pub. No.: US 2004/0053326 A1
(43) Pub. Date: Mar. 18, 2004

(54) Transfer microdessection

(76) Inventors: Michael R. Emmert-Buck, Silver Spring, MD (US); Chad R. Haldeman-Englert, Mesa, AZ (US); Robert F. Bonner, Washington, DC (US); Lance A. Liotta, Bethesda, MD (US)

Correspondence Address: KLARQUIST SPARKMAN, LLP, 121 SW SALMON STREET, SUITE 1600, PORTLAND, OR, 97204, US

(21) Appl. No.: 10/333,374

(22) PCT Filed: Mar. 14, 2001
(86) PCT No.: PCT/US01/08095

(51) Int. Cl. G01N 33/53

(52) U.S. Cl. 435/7.1

(57) ABSTRACT

The present disclosure concerns methods, systems, and devices for analyzing a biological material, such as a cellular or other specimen. In one disclosed example, the method selectively transfers biomolecules from a target region of interest in a biological sample (such as a tissue section). The transfer may occur, for example, by selectively focally altering a characteristic of a transfer layer adjacent the target region, such that the biomolecules can move through the altered area of the transfer layer. In particular examples, the transfer layer is altered by focally increasing a permeability of the transfer layer, for example by removing a focal portion of the transfer layer, and transporting the biomolecules through the altered region of the transfer layer, to microdissect the biomolecules of interest from the biological sample. In yet other embodiments, the microdissected biomolecules can be applied to an analysis substrate containing an identification molecule, such as a nucleic acid array, layered expression scan, or wells containing antibodies. Transfer microdissection allows biomolecules from regions of interest in the biological specimen to be selectively analyzed. For example, nests of highly a typical or metastatic cells in a tumor section can be analyzed for differential expression of certain proteins.

### FIELD OF THE INVENTION

The present disclosure is related to the separation and identification of components of cellular specimens. In particular, it involves analysis of biological specimens, and in particular the analysis of specimen components apart from the remainder of the specimen.

### BACKGROUND

The Human Genome Project and other gene discovery initiatives are dramatically increasing the information available regarding the number, genomic location, and sequences of human genes. Accompanying the expanding base of genetic knowledge are several new technologies geared toward high-throughput mRNA and proteomic analysis of biological samples, allowing a global view of the genes and gene products that reflect normal physiology and pathological states. The expanding genetic database and newly developing analysis technologies hold great potential for increasing the understanding of normal cellular physiology and the molecular alterations that underlie disease states. However, many tissue samples (such as whole cell tissue samples) remain difficult to analyze, given their complex cellular heterogeneity.

Techniques have been disclosed for separating particular subsets of cells from a whole tissue sample. For example, Emmert-Buck et al. (1996) describe the use of laser-based microdissection techniques to rapidly procure microscopic, histopathologically defined cell populations. Examples of laser capture microdissection (LCM) are shown in U.S. Pat. Nos. 5,843,657; 5,843,644; 5,859,699; and 5,598,085, as well as WO 97/13838; WO 98/35216; WO 00/06992; and WO 00/49410, all of which patents and publications are incorporated by reference in their entireties. In LCM, cells of interest are contacted with a transfer member that is selectively and/or focally activated to adhere cells to the activated region of the transfer member. For example, a laser beam can be directed in a microscopic field of view toward the transfer member overlying the cells of interest. The laser beam activates the surface to adhere the cells of interest, and the transfer member can then be removed for further analysis of the microdissected cells. Alternatively, an adhesive layer can be contacted with the cells of interest, and those selected cells are removed from the biological substrate when the adhesive layer is removed.

A more recent approach to the analysis of biological material is layered expression scanning (LES), as disclosed in WO 01/07915, which is also incorporated by reference in its entirety. A biological sample (such as a tissue section) is placed on a layered substrate, in which different layers contain different identification molecules, for example different monoclonal antibodies or nucleic acid probes. Components of the biological sample are then transferred through the layers, by diffusion or electrophoresis, such that different components of the specimen are specifically bound in different layers. The pattern of binding in the different layers can be correlated with the architecture of the biological specimen, to determine different patterns of molecular expression in different regions of the specimen. For example, differences in protein expression can be evaluated between malignant and non-malignant cells in a heterogeneous tumor specimen.

There is still a need for additional methods of analyzing proteins or other molecules of interest present in cellular specimens.

### SUMMARY OF THE DISCLOSURE

The present disclosure describes methods, systems, and devices for analyzing a biological specimen, such as a cellular specimen.

One aspect of the disclosure is a method of transfer microdissection of a biological sample, in which a transfer layer is placed over the biological specimen, and selectively activated over a target region of the sample to increase a permeability of the transfer layer. The increase in permeability can be, for example, a change in the physical and/or chemical properties of the layer that allows biomolecules from the target region to pass through the layer, for example a physical disruption in the layer through which the biomolecules pass, or a focal change in the density of the layer.

Another aspect of the disclosure is a method of analyzing a biological specimen for the presence of one or more biomolecules by contacting a biological sample, such as a tissue specimen, with a selectively activatable transfer layer which can be activated to selectively isolate a target portion of the biological sample. In some embodiments, the target portion of the biological sample that has been isolated is applied to a substrate that includes a plurality of identification molecules, and biomolecules from the target portion of the biological sample are contacted with the plurality of identification molecules to determine if one or more target biomolecules is present in the target portion. The identification molecules can be, for example, specific binding agents, such as antibodies or nucleic acid probes, arranged in an array (such as a microarray) or in multiple contiguous layers (as in a layered expression scan).

The transfer layer can be selectively activated, for example, by adhering the transfer layer to the target tissue to selectively dissect the target tissue from the biological specimen. Alternatively, the transfer layer can be selectively altered, for example by disruption or other change in physical or chemical properties, to allow the target biomolecules to move through the selected area of disruption in the transfer layer. In one specific example, the transfer member is disrupted over a target region of the biological sample by applying radiant energy (such as ultrasonic, thermal, laser or ultraviolet radiation) to the transfer layer to disrupt the layer and selectively open the layer over the target region. The target region can be selected, for example, by microscopic examination of the biological specimen.

The foregoing and other features and advantages of the disclosure will become more apparent from the following detailed description of several embodiments, which proceeds with reference to the accompanying figures. The inclusion of particular examples in this Summary does not imply that they are essential to any aspect of the disclosure.

### BRIEF DESCRIPTION OF THE FIGURES

FIG. 1 is a schematic illustration of a transfer microdissection device, showing a transfer member for selectively transferring biomolecules at targeted locations through holes in the transfer member, using flow of a buffer solution through nitrocellulose membranes.

FIGS. 2A and 2B are schematic diagrams illustrating selective transfer at targeted locations through the holes in the transfer member of FIG. 1.

FIG. 3 is a schematic diagram illustrating how laser microdissection can be used to select a target region (such as a field of cells) in a tissue section, and transfer the selected field to a transfer film. FIG. 3A shows the individual components of the system, while FIG. 3B shows the transfer film in place on the tissue section, with a laser beam focused on the target region to disrupt the transfer film above the target region. FIG. 3C shows a recipient layer in place over the transfer layer, such that biomolecules from the target region can pass through the disruption in the transfer layer, and into the recipient layer.

FIG. 4A is a schematic diagram which shows a microarray having multiple wells with discrete specific binding agents, such as nucleic acid probes or antibodies. FIG. 4B shows the microarry with a transfer layer applied to its surface. FIG. 4C shows the assembly with disruptions provided in the microarray over the wells, to permit selective transfer of biomolecules from an overlying layer that carries a biological specimen.

FIG. 5 is an illustration of a prostate section, showing how different areas of the prostate, and different cell populations, can be targeted for analysis, using transfer microdissection. In this particular embodiment, the method is performed in association with Layered Expression Scanning (LES), in which the capture regions are layers, and they capture proteins or nucleic acids that are present in the specimen.

FIG. 6A is a schematic drawing of a specific disclosed transfer microdissection method. Three different types of starting specimens are shown: a whole mount tissue specimen; dissected, intact cells; and dissected, lysed cells. FIG. 6A also includes an enlarged, perspective view of an example of a layered expression scan substrate, having multiple contiguous porous layers, each layer having a different identification molecule bound within it. FIG. 6B shows the contiguous layers subsequently separated for examination or analysis.

FIG. 7 is a schematic drawing of the use of adhesive microdissection to selectively isolate target cells of interest from a biological specimen, and introduce the isolated cells to an analysis substrate. FIG. 7A shows a tissue section on a glass slide, overlaid with a transfer microdissection layer. FIG. 7B illustrates focal areas of the transfer layer which have been activated to adhere the target regions to the transfer layer. FIG. 7C shows the removal of the transfer layer and the adherent target regions. FIG. 7D shows the application of the target regions to a microarray with wells containing specific binding agents.

FIG. 8 is a schematic view of a transfer layer (in FIG. 8A) applied to a layered expression scan device, in which biomolecules from cellular target regions are retained in different layers of the device (FIG. 8B).

### DETAILED DESCRIPTION OF SEVERAL EMBODIMENTS

Abbreviations

LCM: Laser Capture Microdissection

LES: Laser Expression Scan

### DETAILED DESCRIPTION

This detailed description provides several examples of methods of analyzing a biological sample by placing a transfer member adjacent the biological sample, and activating a discrete portion of the transfer member adjacent a target region of the biological sample, wherein activation of the discrete portion of the transfer member changes a physical characteristic of the transfer member to selectively increase permeability of the transfer member to biomolecules of the target region, such as proteins or nucleic acids. Biomolecules in the target region are then moved through the transfer member for subsequent analysis of the biomolecules. The target regions may be selected, for example using a microscope, and the target region may be an area of biologically distinct cells, such as cells sharing a biological property that is to be studied, such as atypia or dysplasia.

In particular embodiments, activation of the transfer member is achieved by decreasing a density of the transfer member to increase movement of the biomolecules through the transfer member, for example by selectively removing the discrete portion of the transfer member, or selectively increasing a solubility of the discrete portion to at least some of the biomolecules (for example by focally decreasing the density of the transfer member). In some examples, the solubility of the transfer member can be increased to particular proteins of interest. A recipient member can be applied to the transfer member, to retain at least some of the biomolecules that move through the transfer member for detection and/or quantitation. Since the biomolecules move from selected target regions of the biological sample, characteristics of the biomolecules can be correlated with characteristics of the target tissue (or other biological material) from which the biomolecules have been obtained. For example, expression of particular proteins in malignant nests of cells in a tissue specimen section can be detected.

In some examples, the biomolecules are moved through the transfer member in a fluid, such as a buffer liquid that is moved by capillary action or electrophoresis, or a gas that is introduced under pressure. Capillary movement of the liquid can be promoted by placing an absorbent member adjacent the transfer member, in a position that draws the liquid through the biological sample and the transfer member.

Analysis of the biomolecules that are moved from the target region can be performed using a variety of techniques, such as interaction with a specific binding agent, for example an antibody or a nucleic acid. The specific binding agents can be arranged in an array on a substrate, such as a nucleic acid or antibody array or microarray. In particular embodiments, the array is an array of arrays, in which each element of the array includes multiple array elements (such as different cDNAs immobilized on a substrate). Each array in the array of arrays can be identical or different. The transfer member can be interposed between the biological sample and one or more elements of the array, and activated to specifically introduce biomolecules from the target region of the sample into each array, or selected arrays.

Alternatively, the specific binding agents are contained in contiguous layers of an analysis substrate, such as a layered expression scan (LES) substrate, in which different specific binding agents are contained in different contiguous layers of the analysis substrate. Interaction with specific binding agents in one or more of the layers can be correlated with a position of the microdissected sample applied to the substrate, such that the presence of specific biomolecules can be identified in each identified sample.

The following example illustrates one embodiment of transfer microdissection of a biological sample.

### EXAMPLE 1

Transfer Through a Nitrocellulose Barrier

“Transfer microdissection” refers to the selective transfer of biomolecules from target regions in a biological sample out of the sample, through a transfer member which acts as a mask, to selectively allow movement of the biomolecules from the target region, while substantially inhibiting or preventing transfer of biomolecules from other regions of the sample.

In a particular example of this technique, the transfer member is a thin transparent barrier membrane that is placed on a histologic tissue section. The barrier membrane is made of, or includes, a highly concentrated polymer that does not permit standard biomolecules to migrate through it by capillary action, unless the barrier membrane has been altered. While viewing the tissue section microscopically, the barrier membrane is focally altered over the target region, for example by obliteration, such as by manual disruption or laser ablation of the focal area, to form openings through the barrier membrane adjacent the target regions of the biological sample. The tissue section and overlying barrier membrane is then placed in contact with a capture surface or other analysis substrate, and the biomolecules in the tissue section are transferred through the openings and into the analysis substrate. Transfer of the biomolecules from the target region allows a transfer microdissection of molecules from the target region to occur.

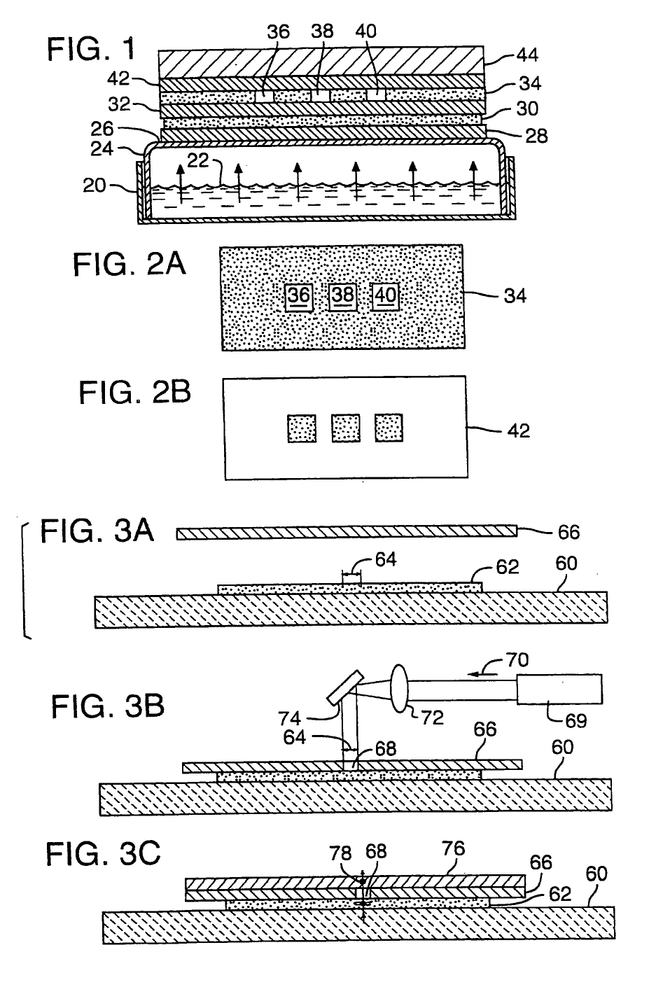

This approach is illustrated in FIG. 1, which shows a container 20 which contained 500 ml of 1×Tris-Glycine Buffer solution 22. An absorbent support 24 is placed in container 20, to form a support surface 26 through which the liquid buffer flows. A layer 28 of 2% agarose gel is pipetted on to surface 26, and a 10μ thick tissue section 30 is placed on top of gel layer 28. A layer 32 of 1% agarose gel is pipetted on to tissue section 30. An unblocked nitrocellulose membrane 34 (1 inch×0.75 inch×100μ thick, 0.40 μm pore size, from Schleicher and Schuell, Keene, N.H., product #BA-85) cut to the width and length of layer 32, was placed on top of agarose layer 32. Nitrocellose membrane 34 had three square holes 36, 38 and 40 cut in nitrocellulose layer 34, each of the holes being 1.5 mm×1.5 mm in size.

An unblocked nitrocellulose membrane 42 (without holes in it) was placed on top of nitrocellulose layer 34 to serve as an analysis substrate, which was in turn overlaid with blotting paper 44 to increase the flow of buffer liquid through the apparatus. The liquid was allowed to flow for four hours to transfer biomolecules from tissue section 30, through holes 36, 38 and 40.

FIG. 2A is a depiction of nitrocellulose membrane 34 with holes 36, 38 and 40 cut in it. FIG. 2B shows nitrocellulose analysis layer 42. Both layers 34 and 42 were stained with Ponceau's stain for 30 minutes to detect proteins, and then destained with distilled water for 30 minutes to remove stain from areas in which protein was not present. The unblocked nitrocellulose layer 34 retained most of the tissue proteins (as illustrated by the dark staining in layer 34 of FIG. 2A). However, proteins from the tissue section underlying the holes in the first nitrocellulose layer passed to nitrocellulose layer 42 where they were bound (the dark squares in layer 42 correspond to proteins that moved through transfer pathways 36, 38 and 40).

### EXAMPLE 2

Laser Assisted Transfer Microdissection

Laser assisted microdissection is illustrated in FIG. 3, which shows in FIG. 3A a glass slide substrate 60 on which is placed a 10μ thick tissue section 62 having a selected field of cells of interest 64 (such as cells exhibiting cellular atypia). Tissue section 62 is then covered with a nitrocellulose transfer member 66, as shown in FIG. 3B. A laser 68 acts as a source of radiant laser energy, and directs a beam 70 of collimated light towards a lens 72, which directs the beam toward a directional mirror 74. The position of mirror 74 is adjusted to direct beam 70 on to transfer member 66 above the selected field of cells 64, to ablate a portion of transfer member 66 above cells of interest 64. The laser beam can be selected to be of any size or shape, but in this particular example the beam is circular in cross section, and 5-50μ wide, so that it ablates a cylindrical hole 68 of similar width through transfer member 66 above field of cells 64.

A recipient member 76 in the form of a thin nitrocellulose layer (FIG. 3C) is then placed on top of transfer member 66, and transfer of cells 64 is accomplished by electrophoresis or capillary action. A permeable gel (such as an agarose gel) could be placed between recipient member 76 and transfer member 66 to further facilitate transfer of biomolecules. The biomolecules are moved from the field 64 into recipient member 76, as illustrated at 78 in FIG. 3C. In the case of capillary transfer, the illustrated recipient member is 100 μm thick, 0.40μ pore size, from Schleicher and Schuell, Keene, N.H., product #BA-85. The specific chemical composition or pore size of recipient member 76 is not critical, as long as it is capable of absorbing and retaining the biomolecules that are transferred to it.

The characteristics of the laser beam 70 can be selected from a variety of options to achieve ablation of the transfer member 66 and form hole 68. In particular examples, the laser applies sufficient energy to precisely ablate an opening of the desired diameter through transfer member 66. The size of the hole is selected by the diameter of the laser beam that is applied; the diameter of the beam is approximately the same as the area of field of cells 64 that is selected for analysis. The energy selected for the applied beam depends on the substrate through which the hole is formed. For example, for a thin polymer layer, a focal 2μ spot at up to 350 nm wavelength is used. An example is a nitrogen laser, pulsed at 337 nm wavelength. For a 1μ thick transfer layer, absorption would (for example) be selected to be attenuated at 0.33μ depth, so that 3 pulses (for example at about 0.5 mJ) would precisely ablate a hole through transfer member 66. The wavelength of the laser could also be selected to be different than a wavelength at which the tissue in section 62 would absorb significant energy, to avoid photochemical degradation of the tissue specimen that is to be analyzed. In some examples, a thin polymer gel layer (such as an agarose gel layer) is placed between transfer member 66 and tissue section 62 to provide additional protection for the underlying tissue section.

Although layers 62/66 and 60/62 are shown in direct apposition to one another, it is helpful in some examples to place a layer of porous hydrophilic gel matrix between layers 62/66 and 60/62 to improve contact between the layers (since microscopic imperfections in the surfaces of the contiguous layers could affect movement of the biomolecules).

### EXAMPLE 3

### Transfer Microdissection with Layered Expression Scan Analysis

Transfer microdissection can be combined with any of a variety of analytic techniques, including high-throughput analysis of nucleic acids and proteins, for example antibody and nucleic acid arrays. However, the invention is not limited to any particular analytic technique, and for purposes of illustration only, will be described in this Example in connection with Layered Expression Scanning (LES). More detail about LES can be found in WO 01/07915, which has been incorporated by reference in its entirety.

In a particularly discussed embodiment, biological specimens (such as tissue sections or other cell populations, which are referred to herein as cellular specimens) may be separated into multiple layered substrates, such that each of the layers can be subjected to a separate analysis that can be correlated with the architecture (such as the cytological architecture) of the original specimen, or the position of an opening in the transfer layer through which biomolecules are transfer microdissected out of a biological sample.

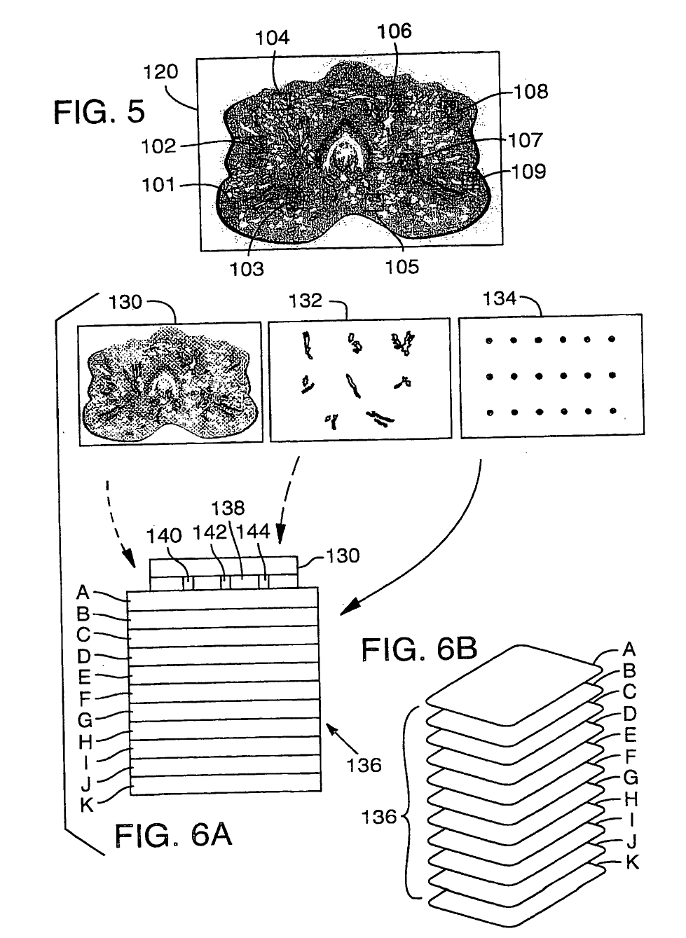

The prostate tissue section of FIG. 5 illustrates how intact tissue sections may have different microscopic variations, which can be usefully correlated with the results of the different analyses. FIG. 5 shows a section of prostate tissue 120, having an area 101 of lymphocytes not associated with tumor; area 102 of normal epithelium, adjacent to tumor; area 103 of low grade tumor; area 104 of stroma; area 105 of high grade tumor; area 106 of hyperplasia; area 107 of low grade prostatic intraepithelial neoplasia (PIN); area 108 of normal epithelium, not adjacent to tumor; and area 109 of lymphocytes, associated with tumor. It is of interest to be able to determine different molecular characteristics of the intact tissue specimen, and correlate those molecular characteristics with particular regions of the tissue. Particular embodiments of the layered expression scans (LES) of the present invention make this possible.

One example of a layered expression scan is shown in schematic form in FIG. 6. One or more biological samples, such as an intact tissue section (for example prostate section 130), dissected intact cell lysates 132, or dissected cell lysates 134, are prepared and placed within or upon an ultra thin gel, called a sample gel, which is applied to a multilayered substrate, for example to the top of the multilayered substrate 136. For purposes of illustration, FIG. 6A shows tissue section 130 in the ultra thin gel, applied to a top surface of the multilayered gel 136. The sample gel can utilize any known gel matrix including agarose, polyacrylamide and gelatin based matrices. If the sample gel is agarose, its concentration is, for example, in the range of about 0.1% to about 5%, and it may be cast to be “ultrathin,” that is, in the range of about 0.10 μm to about 1 mm thick.

Tissue section 130 (or the gel containing it) is placed on top of a transfer layer 138 (such as a nitrocellulose membrane) having focal openings 140, 142 and 144 that are aligned with structures of interest in tissue section 130 (such as areas 103, 105 and 109 shown in FIG. 5). The alignment of openings 140, 142 and 144 biomolecules from the selected structures of interest to migrate through the transfer layer 138, but inhibits or prevents migration of biomolecules from the portions of tissue section 130 that are not aligned with the openings.

Transfer layer 138 is placed on the top surface of the substrate layer A, which surface is substantially parallel to separations between the layers. For purposes of illustration, eleven layers are shown (although many more can be used, for example at least hundreds or thousands of layers), and the layers are labeled A through K. Each of the layers may be a membrane or film, each of which may contain one (or more) identification molecules, such as an antibody that recognizes a particular antigen, or a DNA sequence that functions as a probe by hybridizing to complementary DNA sequences in the specimen. The identification molecule can be different in each of the layers A-K or the same.

The soluble contents of the specimen aligned with openings 140, 142, 144 are transferred (for example by capillary action or electrophoresis) through openings 140, 142, 144 and through the series of layers A-K, while maintaining the overall two-dimensional relationship of the holes 140, 142 144, as well as retaining cellular architecture (if any) within the sample. The location of the biomolecules in the layers A-K will therefore correspond to the locations of the openings 140, 142 and 144 through which the biomolecules passed to move into the layers. As the specimen components, such as proteins and nucleic acids, pass through the membranes, the identification molecules of the substrate layers interact with the proteins or molecules of interest. After this interaction occurs, the membranes are separated (FIG. 6B) and may be subjected to further analysis, such as exposure to a second antibody or DNA sequence, producing a highly sensitive and specific molecular profile, or “expression scan” of the cellular specimen. The presence of identified biomolecules in each layer can be correlated with the position of an opening in transfer layer 138, to determine in which target region of the biological specimen this biomolecule was present.

If the analysis is made with respect to a whole tissue specimen, the first step of the method can involve examination of a reference specimen cut from a location immediately adjacent to the first tissue specimen, so that areas of interest in the intact specimen (such as areas of cellular atypia) can be identified, and openings formed in the transfer member to align with the structures of interest. In this manner, molecular characteristics of the specimen (such as the expression of particular proteins) can be correlated with areas of histological interest (such as invasion of the prostate capsule). For example, expression of particular proteins associated with capsular invasion (or metastasis in general) can be located.

In particular examples, the biological sample is a cellular specimen. Cellular specimens include, but are not limited to, tissue sections, cultured cells, or a cytology sample. Tumor tissue sections produced by the cryostat method are particularly suited for use in the present method. Standard methods of preparing tissue sections are taught in Lefkovits et al. (1996). If the molecule of interest is present at moderate or low level abundance, such as those present in the range of one to 10,000 copies per cell or even one to 100 copies per cell, the thickness of the tissue section to be analyzed can be increased to intensify the expression scan produced. The thickness of such samples are about 25 μm to about 50 μm. Since an adjacent reference specimen may be used to view the tissue microscopically, and the sections are thin, the histological detail of the analysis is not compromised by utilizing the thicker tissue section for the present method.

The sample gels in which the biological specimen is optionally placed improve ease of handling prior to analysis. The gel can be, for example, an ultra thin gel made of agarose or polyacrylamide. Alternatively, the gel can be used as the transfer layer (e.g. instead of the nitrocellulose layer). The sample gel could be made using standard 2% agarose dissolved in tris-borate EDTA buffer. Two hundred μl of this preparation is pipetted onto a standard glass histology slide and coverslipped, thus creating an ultrathin gel on the order of 0.5-1 mm thick. The sample gel can be selected to participate in separating the different components of the cellular specimen, by altering a chemical characteristic of the gel (instead of, or in addition to, providing openings in the transfer member).

This separation function is accomplished by providing the sample gel with a particular structure that alters or aids the migration of certain components into the layers of substrate 136, and/or retards the migration of components that should remain in the sample gel. Structural changes that aid the separation function include varying the gel concentration to alter the gel pore size, or varying gel composition, such as using an acidic or basic formulation to aid or retard the migration of certain components. If no separation function by the sample gel is desired, a gel with neutral characteristics can be chosen, such as 2% agarose in TBE with a pH of 7.4.

Even if a gel is not used, the analyzed cellular specimen can be treated before transfer to allow selective transfer of certain target molecules into the substrate layers. An example of such a treatment is the use of a transfer buffer that contains detergents, which would tend to increase the transfer of components of the cellular specimen that are present in the cellular membrane (such as the plasma membrane).

If the samples are solubilized cellular lysates, purified proteins, or nucleic acids, it is possible to prepare a sample gel as follows. A 2 mm thick 2% agarose gel is “punched” to generate a series of holes (4 mm in diameter, for example) that serve as sample “wells.” The samples may then be added to 1% liquid agarose, placed into the wells, and then allowed to solidify to form a sample gel 134. The sample gel created by this process may then be placed on top of the transfer member 138, aligned with the holes through the transfer member. In this manner, only the samples are aligned with the holes, such that no stray biomolecules are transferred through the separation layers.

The layered substrate 136 of the embodiment disclosed in FIG. 6A includes separable layers of a material (such as layers A-K of nitrocellulose, which can be obtained from Schleicher and Schuell, Keene, N.H., product #BA-85) which is capable of placement in multiple contiguous layers, as shown in FIG. 6A, and subsequent separation into multiple separate (non-contiguous) layers, as shown in FIG. 6B. Once the components of the specimen have migrated through the contiguous layers, the layers are separated to permit individualized analysis of the components of the cellular specimen retained in each separated layer.

Other examples of the substrate layers include, but are not limited to high concentration agarose gels, low concentration agarose gels, high concentration polyacrylamide gels, a low concentration polyacrylamide gel, and membranes, such as porous membranes like nitrocellulose paper. Low concentration agarose is from about 0.1 to about 3%, while high concentration is above about 3%. Low concentration acrylamide is about 2% to about 20%, while high concentration is above about 20%. Such gels or membranes may optionally be backed with a polyester membrane or the like to provide mechanical strength and to provide a “contact substance” that permits efficient transfer of the components of the cellular specimen between the layers of the substrate and reduces loss of the two-dimensional architecture of the sample as the cellular components migrate through the substrate 136.

Nitrocellulose layers are examples of porous layers that can be used in layered expression scanning devices, to exert capillary pressure on a specimen (such as specimen 130) on the top surface of transfer member 138 (FIG. 6A), and conduct components of the specimen through the layers. Such porous layers or membranes allow the movement of liquid from one face to an opposite face of the membrane, and exert capillary action on the specimen to move soluble components of the specimen through the multiple layers. Although nitrocellulose avidly binds biomolecules such as proteins, the nitrocellulose can be altered with well known blocking agents to inhibit e.g. protein binding, and promote movement of the protein or other biomolecule through the nitrocellulose layers.

Blocking agents serve to prevent non-specific interactions between the substrate layer and components of the sample as they are transferred through the substrate. “Blocking agent” is a collective term for various additives that prevent non-specific binding, but that have no active part in the specific reaction, such as an immunochemical reaction, between a particular identification molecule and its target. Blocking agents are most commonly concentrated protein solutions. Examples of such solutions include 10-20% fetal calf serum and 5% non-fat dry milk powder dissolved in a buffer such as PBS, TBS, or TBST. Commercially available blocking agents include SuperBlock™, Blocker™ BLOTTO, Blocker™ BSA, and SeaBlock™ (Pierce Chemical, Rockford Ill.) as well as NAP-SureBlocker™, a non-animal protein blocking agent (Geno Technology, Maplewood, Mo.).

The pore size of the porous layers may be any that are available, particularly the about 0.45 μm pore-size nitrocellulose membrane. The number of layers in the substrate can vary widely, for example from about 1 to at least 2, 5, 10 or even 1000 layers, although for purposes of illustration eleven layers A through K are shown in FIGS. 6A and 6B. The number of layers can be varied, depending in part on the number of different binding or other identification molecules being used, and is ultimately limited only by the ability to promote migration of the cellular components through the substrate levels. The substrate layers can be of identical structure, or the layers can be mixtures of different substrate types.

In a disclosed embodiment, each layer (or other type of region) of the substrate is impregnated with multiple copies of at least one identification molecule that can interact with one or more molecules of interest. Similarly, different layers of the substrate can contain multiple different identification molecules, for example each layer (or other type of region) can have one or more identification molecules present. In an alternative embodiment of the substrate, all the layers (or other type of region) would contain the same identification molecule and differential migration through the various substrate layers would allow separation. The differential migration can be promoted by differing physical characteristics of the substrate layers, such as different pore diameters or pH, or porosity or pH gradients, in the direction of layers A to K. Likewise, in other embodiments, some of the substrate layers do not contain identification molecules and may serve to promote differential migration of sample components through the layers.

Representative examples of identification molecules include, but are not limited to antibodies, nucleic acids, peptides, receptors, ligands, dyes, stains, or colorimetric enzymes. Specific examples of identification molecules include anti-prostate specific antigen antibodies (Scripps, San Diego, Calif.; anti-cytokeratin antibodies, anti-alpha-actin antibodies (Sigma, St. Louis, Mo.); anti-PB39 antibodies, and anti-menin antibodies (National Cancer Institute Core Antibody Lab, Fredrick, Md.). Identification molecules can interact specifically with the molecule of interest, such as the binding of an antibody or complementary interaction with a single stranded DNA sequence, or more generally, such as the interaction between a dye and a molecule colored by that dye. If the identification molecule prevents the migration of the molecule of interest into subsequent layers of the substrate, the identification molecule is referred to as a capture molecule.

When the transfer of the components of the cellular specimen occurs through capillary movement of liquid present in the sample through the substrate, it is desirable to have the multiple layers (or other regions) of the substrate in physical contact with each other. The use of contiguous substrate layers A-K (as in FIG. 6A) reduces the effects of diffusion on the accurate migration of the proteins or molecules of interest through the substrate and enhances the capillary movement of the components. Alternatively, the components can be moved through the substrate layers (or other regions) using electrophoresis, a variation of isoelectric focusing, or other similar methods of moving charged molecules. If electrophoresis or another method using electricity is used, the different layers of the substrate are ideally conductive, such as an agarose or polyacrylamide gel. Methods based on electrophoresis would be limited generally to separation of charged species from the cellular specimen. However, the use of electrophoresis can avoid the use of contiguous substrate layers. For example, the layers could be separated from one another, as long there is an electrically conductive medium (such as a liquid, particularly a liquid comprising ions, such as may be formed by dissolving a salt in a liquid) between the layers through which the specimen is electrophoresed.

Another means of transferring sample components through the substrate layers (or other regions) is by way of liquid movement in response to a fluid pressure differential. For example, pressure, such as provided by a compressed gas, may be applied to the sample to force the liquid present in the sample into and through the substrate 136. Alternatively, another liquid under pressure may be used to carry sample constituents into and through the substrate layers to an area of lower pressure. Liquid present in a sample or provided to carry sample constituents into the substrate layers may also be induced to move through the substrate 136 by a vacuum applied to the substrate 136.

### EXAMPLE 4

### Selective Transfer of Tissue Section Contents for Layered Expression Scanning

This example discloses selective transfer (transfer microdissection) from a tissue section to a substrate, such as a Layered Expression Scan (LES) stack, wherein the LES stack containing 10 layers is prepared as described in Englert et al., Cancer Research, 60: 1526-1530, 2000. The first nine layers of the stack are nitrocellulose layers treated with a commercial nitrocellulose blocking agent and the tenth layer contains anti-PSA (prostate specific antigen) antibodies. A prostate section, embedded in an ultrathin gel of 2% agarose and placed on a slide, is examined microscopically to determine the relative positions of separate populations of epithelial cells and non-epithelial cells within the section. Two 1 mm holes are punched through an unblocked nitrocellulose membrane (the transfer member) at positions that will correspond to the observed locations of the separate populations of epithelial and non-epithelial cells when the nitrocellulose membrane is placed squarely over the slide bearing the entire tissue section. The nitrocellulose transfer member is then properly positioned on the slide and adjusted to ensure that the holes correspond to the location of the desired cell populations. Once the nitrocellulose transfer member is properly positioned, the tissue section and the nitrocellulose transfer member are removed from the slide and placed in contact with the LES stack so that the transfer member is between the tissue section and the LES stack. Following transfer of the contents of the tissue section through the LES stack by capillary action, the LES layers are separated and then first probed with monoclonal anti-PSA antibodies (Scripps, San Diego, Calif., 1:1000 titer) and then a second time with a biotinylated secondary antibody (Sigma, 1:5000 titer) for 30 minutes at room temperature, followed by visualization by enhanced chemiluminescence.

Only the tenth layer of the LES stack will show a signal indicating the presence of captured PSA in that layer and, within the tenth layer, PSA will only be detected at a position corresponding to the hole in the nitrocellulose membrane that was placed over the population of epithelial cells in the tissue section. This result is consistent with the known epithelial localization of PSA.

In other embodiments, holes may be introduced into a nitrocellulose transfer member using focused pulses of laser light. For example, an excimer laser, such as a XeF or KrF excimer laser, that is directed along the optical path of a microscope may be used to selectively ablate a nitrocellulose barrier member to form holes at positions identified during inspection of the underlying tissue section and identified by their stored microscope stage positions. Such an operation may optionally be performed robotically.

Alternatively, the nitrocellulose membrane may be treated with xylenes to facilitate observation of the tissue section underneath the nitrocellulose membrane, thereby enabling simultaneous observation and hole creation. In this particular embodiment, the holes in the nitrocellulose membrane may for example be created by manual removal of the overlying nitrocellulose membrane using microsurgical tools.

In yet other embodiments, the transfer member may be a gel of either agarose or polyacrylamide into which a laser light absorbing molecule is impregnated. The laser light absorbing molecules may either be dispersed throughout the gel as a suspension or dissolved therein. Light absorbing molecules, such as phthalocyanines and naphthalocyanines, for example vanadyl 2,11,20,29 tetratert-butyl-2,3-naphthalocyanine, that efficiently absorb near-infrared laser light, serve to convert the laser energy into heat energy that vaporizes the gel over the targeted portions of the tissue section. Transparent gels allow holes to be introduced into the transfer member while the tissue section is under microscopic observation.

In still other embodiments, the transfer member is a polymer membrane that inhibits the transfer of the contents of a biological sample but is capable of conducting buffer solutions into and through the LES stack. The transfer member in this embodiment is also capable of being selectively altered or ablated by laser radiation. Such a membrane may also include molecules, such as dyes, that absorb laser radiation and facilitate ablation of the membrane.

### EXAMPLE 5

### Targeting Biomolecules to a Substrate

Another aspect of the disclosure is a method of allowing targeted biomolecules to move from a biological specimen and interact with an analysis substrate, such as a microarray. This can be particularly useful in situations in which a small number of cells are available for analysis, for example if a small number of cells have been microdissected from a larger population of cells (such as a tissue specimen). In some embodiments, a transfer member is utilized to selectively transfer components of a biological specimen for subsequent analysis using a microarray.

Microarrays (also known as “biochips”) are microscopic arrays of immobilized identification molecules, such as nucleic acids, peptides, receptors, ligands, dyes, stains, or colorimetric enzymes, for example, antibodies, cDNAs, and oligonucleotides.

Patents and patent applications describing arrays of biopolymeric compounds and methods for their fabrication include: U.S. Pat. Nos. 5,242,974; 5,384,261; 5405,783; 5,412,087; 5,424,186; 5,429,807; 6,436,327; 5,445,934; 5,472,672; 5,527,681; 5,529,756; 5,545,531; 5,554,501; 5,556,752; 5,561,071; 5,599,895; 5,624,711; 5,639,603; 5,658,734; 6,087,102; WO 95/21265; WO 96/31622; WO 97/10365; WO 97/2727317; EP 373 203; and EP 799 897.

Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; WO 95/21265; WO 96/31622; WO 97/10365; WO 97/27317; EP 373 203; and EP 785 280.

References providing a review of micro array technology include Lockhart et al., Nature Biotechnology, 14: 1675, 1996; Epstein and Butow, Current Opinion in Biotechnology, 11: 36-41, 2000; Khan et al., Biochimica et Biophysica Acta, 1423: M17-M28, 1999; and Watson and Akil, Biol. Psychiatry, 45: 533-543, 1999. A review of the technology may also be found on the “Gene Chips” website.

In a particular embodiment of selective transfer for subsequent microarray analysis, cells are laser capture microdissected using a water permeable or permeablized thermoplastic capture membrane. Subsequent to adhering cells, such as tumor cells, to the thermoplastic membrane, the contents of the microdissected cells are transferred (in any manner described above, such as by capillary action) to a small microarray that is in the form of a small well that is about 10-300 μm in diameter, such as about 100 μm in diameter, that has immobilized at its bottom surface a number of identification molecules arranged in an array. Many different identification molecules can be arrayed within the well, for example from 1 to about 100, such as from about 20 to about 80. The microarray at the bottom of the well may then be probed (for example with fluorescent secondary antibodies) to reveal the presence of molecules transferred from the specimen.

Such small microarrays allow analysis of the components of a small number of microdissected cells, such as from one cell to about 100 cells, for example from one cell to about 50 cells. Direct transfer of the contents of a population of rare cells to a very small microarray reduces the dilution of cellular contents and may be especially useful for clinical analysis thereof. Analysis of cells in this manner for a small number of components, such as from 1 to about 100, also increases the speed at which analysis may be accomplished, as the dimensions of the microarray are small and diffusion takes place quickly over such distances.

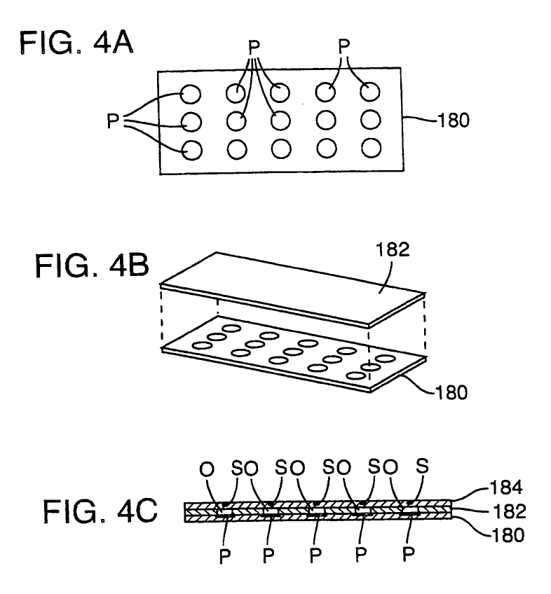

Targeting of biomolecules to an analysis substrate can be combined with transfer microdissection, as shown in FIG. 4, which illustrates use of a selectively activated transfer member to allow certain target biomolecules in a specimen to interact with specific binding agents on a substrate.

FIG. 4A shows a substrate 180, such as a nucleic acid microarray chip with a plurality of cDNA probes adhered in discrete spots P to the chip in a known manner. Alternatively, the substrate could be a plate with a plurality of microtiter wells, but for purposes of explanation this example will be described in connection with a cDNA array. As shown in FIG. 4B, substrate 180 is overlaid with a transfer member 182, such as a film that is capable of providing selective openings through transfer member 182, wherein each opening O (FIG. 4C) communicates with only one of the probe spots P on the array. The openings can either be preformed in alignment with the array spots, or formed by selective focal activation of transfer member 182, for example by focal exposure of member 182 to radiant energy, such as laser, infrared, or ultraviolet energy that changes a physical property of member 182 to allow biomolecules of interest to pass through the member. The opening O need not be a physical opening, but can instead be a functional opening, in which chemical or physical properties of member 182 have been focally altered to allow the biomolecules to move through member 182.

FIG. 4C illustrates a specimen layer 184 (such as an agarose gel layer) which is applied over transfer member 182, with biological samples S in layer 184 aligned over respective openings O that are in turn aligned with probe spots P. Samples S can be, for example, microdissected cells that are placed over the openings O and respective probe spots P to most efficiently use a small number of cells for analysis. Biomolecules from the cells can be transferred, for example by capillary action or electrophoresis, from specimen layer 184, through one of the openings O in transfer member 182, and into a probe spot P for interaction with cDNA probes that recognize mRNA from the cells. Alternatively, the spots can contain antibodies that specifically bind to proteins from the cells.

### Example 6

### Preparation of Transfer Members Incorporating a Foamant

As already noted, openings can be made in transfer members by mechanical (for example a punch) or other ablative means (for example using a laser). It is also possible to prepare transparent thermoplastic polymer transfer members, which have incorporated in them a blowing agent, also known as a foamant. The blowing agent serves to permeabilize the transfer member by producing a gas that creates an open cell structure through the transfer member. Blowing agents may be activated by heat, such as the heat generated by absorption of laser pulses. If the transfer member is otherwise impermeable to the contents of a biological sample, a functional “hole” may be created by foaming the transfer member at a particular position, for example by locally heating the layer with a focussed pulse of laser light.

Polymer transfer members, especially thermoplastic resins, incorporating a blowing agent may be prepared according to the methods disclosed in European Patent 345,855. Briefly, unreacted blowing agents that are also solvents for the polymer may be incorporated by exposing the polymer directly to the liquid blowing agent or alternatively by exposing the polymer to the vapor of the blowing agent. Solid blowing agents may be incorporated by exposing the polymer to a polymer solvent containing dissolved blowing agent. Suitable blowing agents include both physical and chemical blowing agents.

Examples of physical blowing agents include, among others, di-, tri- and tetrachloromethane, trichloroethene, 1,2-dichloroethane, lower hydrocarbons, such as butane, different pentanes, hexanes, and heptanes etc., which also comprise the different isomers thereof, cyclic aromatic and aliphatic hydrocarbons etc., but also lower alkanols, ethers and ketones. It is also possible to use chlorofluorocarbons, but their use is discouraged for environmental reasons.

Examples of chemical blowing agents include sodium bicarbonate, azodicabonamide, azobisisobutyronitrile, diazoaminobenzene, p-toluenesulfonyl kydrazide, benzenesulfonyl hydrazide, dinitrosopentamethylenetetramine, oxybis(p-benzenesulfonyl)hydrazide, N,N′-dinitroso N, N′-dimemethyleterepthalamide, p-toluenesulfonyl semicarbazide, 5-phenyltetrazole, and others. These compounds can be used as such or in combination with an activator. Examples of activators are zincoid, metals, salts of sulfonated compounds, activated urea, stearic acid, and polyethylene glycol, among others. Activators serve to lower the temperature at which the blowing agent decomposes to generate gas.

The blowing agent may be added to the polymer in an amount from about 0.5% based upon the weight of the polymer to about 50% by weight, such as from about 1% by weight to about 50% by weight, for example, from about 5% by weight to about 50% by weight.

The exact amount and choice of blowing agent needed and the necessity of adding an activator will depend upon the maximum temperature that can be tolerated by the underlying biological specimen as well as the identity of the polymer forming the barrier membrane. Also, the softness of the polymer itself when heated to its maximum will help determine the amount of blowing agent necessary to generate an open-cell, permeable “hole” over selected portions of the tissue sample. Additional examples of blowing agents and their properties as well as a guide to selecting the proper blowing agent for a particular polymer are found at the Uniroyal Celogen® website.

A laser light absorbing, heat generating dye, such as a naphthalocyanine dye, may also be incorporated into the membrane to increase the temperature to which the polymer layer is heated when struck with a laser pulse of suitable wavelength.

### EXAMPLE 7

### Other Transfer Members

In some embodiments, selective transfer of components from a tissue section to the substrate, such as the layered LES substrate, may be accomplished by covering a tissue section with an impermeable or semi-permeable photodepolymerizable polymer, photodepolymerizing the polymer over desired portions of the tissue section, removing the photodepolymerized polymer to create “holes” over the cells of interest, and transferring the contents of the cells of interest into the substrate.

Methods for selectively depolymerizing a polymer layer over desired cells are disclosed in U.S. Pat. No. 6,087,134 to Saunders. Photodepolymerizable polymers include quinone diazides, novalak resins, and acrylics. Additional materials that are photodepolymerizable are disclosed in C. G. Roffey, Photopolymerization of Surface Coatings, John Wiley & Sons, 1982 and in W. Schnabel, Polymer Degradation, Hanser Int., 1981.

Photopolymerizable polymer materials may also be utilized to selectively protect the contents of cells of interest from being transferred in a first transfer to, for example, an unprotected nitrocellulose membrane. Alternatively, the unprotected portions of the tissue sample may be subjected to a degradative enzyme, such as a proteases and nucleases to remove proteins and nucleic acids from all the unprotected cells. The protective polymerization may be accomplished according to the methods of U.S. Pat. No. 6,087,134 to Saunders. In a particular embodiment, selective photopolymerization may be accomplished with a laser directed along the optical path of a microscope, so that visualization and protection occur together. Materials suitable for this application are disclosed in U.S. Pat. No. 6,087,134 to Saunders, C. G. Roffey, Photopolymerization of Surface Coatings, John Wiley & Sons, 1982 and in W. Schnabel, Polymer Degradation, Hanser Int., 1981.

Subsequent depolymerization and removal of the protective polymer layer from the cells of interest may be performed, for example, with actinic radiation or by chemical means. Transfer of the contents of the cells of interest into an LES substrate for analysis then follows removal of the protective polymer layer.

### EXAMPLE 8

### Analysis of Transfer Microdissected Biological Material

A more efficient, targeted use of cellular material for analysis can be performed using techniques other than the targeted disruption or change in other physical characteristic of a transfer member. For example, transfer microdissected cells or biomolecules can be moved from a biological sample, into contact with an analysis substrate, so that a relatively few number of selected cells are analyzed. Examples of this technique are microdissection of the cells by adhering a target region of the transfer member to the biological sample (such as a tissue section), and isolating the region by separating the transfer member from the biological sample while maintaining adhesion with the activated region of the transfer member, so that the at least one portion of the biological sample is extracted from a remaining portion of the biological sample and exposed to the analysis substrate. Alternatively, a region of a transfer member can be removed or altered to allow biomolecules from the biological specimen to selectively move through the transfer member into contact with the analysis substrate.

The analysis substrate to which the transfer member is then applied may include many different analysis stations, and each station can further include a plurality of identification molecules, such as different nucleic acid molecules, for example arranged in separate nucleic acid arrays on a surface of the substrate. The separate nucleic acid arrays may be identical or different arrays. Other examples of identification molecules include antibodies, such as monoclonal antibodies, which identify a specific binding partner. The different antibodies can identify the same or different binding partners.

In particular embodiments in which the analysis substrate includes a plurality of different layers, different identification molecules are contained in at least some of the different layers. In other embodiments, the analysis substrate includes a plurality of chambers or nucleic acid arrays, for example in which each of the plurality of chambers or arrays is no greater than 300 μm wide, and no more than 100 cells are introduced into each well or array.

In a particular example disclosed herein, a transfer member is altered to allow the selective transfer of target biomolecules to the analysis substrate. The transfer member is altered, for example by fusing the targeted locations of the biological specimen to the transfer member, and then exposing the fused targeted locations to the surface of the analysis substrate. The targeted locations can be fused to the transfer member by localized application of heat or other radiant or electromagnetic energy, for example by performing laser capture microdissection of the biological specimen to transfer components of the targeted region (such as selected cells that share a particular characteristic, such as dysplasia or metaplasia) to a transfer membrane. The transfer membrane is then placed in contact with the surface of the substrate for subsequent transfer of the targeted components to the substrate. Alternatively, other forms of microdissection can be used (such as adhesive microdissection of the kind shown in U.S. Pat. No. 5,843,644, which patent is incorporated in its entirety herein).

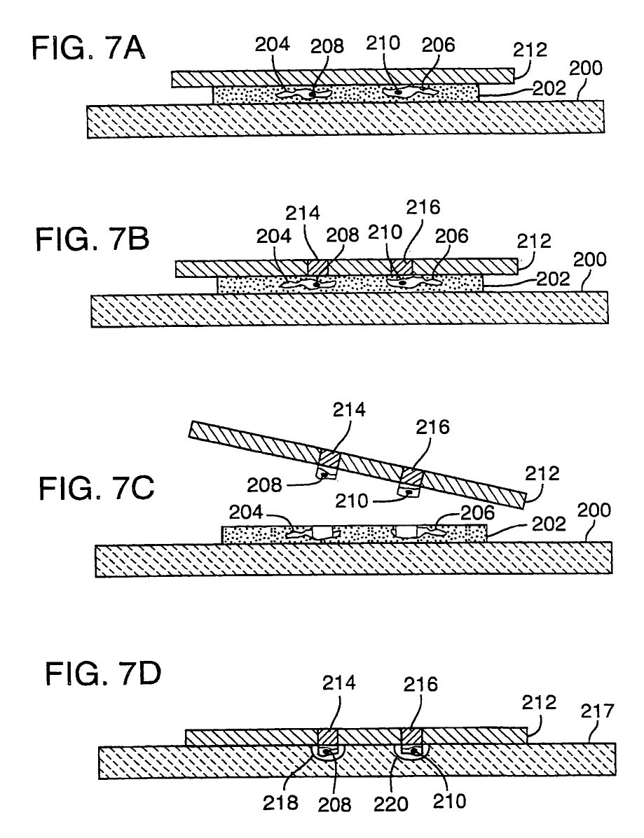

An example of this approach is shown in FIG. 7, in which FIG. 7A illustrates a substrate 200 (such as a glass slide) which carries a tissue section 202 having cellular regions 204, 206 with target regions of interest 208, 210 (such as areas of cellular atypia). A transfer member 212 (such as an adhesive microdissection transfer member of the type shown in U.S. Pat. No. 5,843,644) is applied to tissue section 202. Focal regions 214, 216 of transfer member 212 are then activated, for example by the application of pressure, or of heat as shown in U.S. Pat. No. 5,843,644, to fuse the focal regions 214, 216 to the target regions of interest 208, 210. As shown in FIG. 7C, transfer member 212 is then peeled off or otherwise removed from tissue section 202 and substrate 200, while focal regions 214, 216 are adherent to the target regions 208, 210, which selectively removes the target regions from the tissue section 202.

Transfer member 212 can then be applied to a microtiter plate 216 (FIG. 7D) or other analysis substrate (such as a nucleic acid array), with the target regions 208, 210 aligned respectively with capture regions, such as wells 218, 220. In this manner, the biomolecules in the target regions of interest 208, 210 can be efficiently analyzed, relatively free of background analytes that could potentially affect the analysis. Moreover, this technique is a fast and efficient way to select target regions, and analyze biological molecules from the region, in a manner that will more reliably reveal differential expression of biomolecules in the target region.

Examples of cellular specimens that can be analyzed include, but are not limited to, tissue sections (particularly tumor tissue sections), a cytology sample, microdissected cells and cultured cells. Crytostat tissue sections cut slightly thicker than usual, that is about 25 to about 50 μm, improve the detection of molecules of moderate and low level abundance. Transfer microdissection of targeted regions of the tissue sections can be performed using the methods and devices disclosed herein.

Examples of capture regions of the analysis substrate, in addition to the wells 218, 220 in FIG. 7C, include other chambers, matrices or layers, such as the layers of a layered expression scan. The capture regions can range, for example, from about 1 to more than a hundred, for example several hundred, several thousand, or several tens of thousands in number. In specific examples where the region is a layer with a capture molecule, the chamber has a thickness (for example) of at least about 25 nm. In particular embodiments, the regions may extend across the substrate (as in layers), and components of the specimen are transferred generally transverse to the layers, but they may be transferred substantially parallel or at other angles to the layers. Identification molecules present in the substrate layers may, for example, be antibodies that interact with the components of the cellular specimen, and can be used to identify particular molecules of interest present in the specimen. Other representative, non-limiting examples of identification molecules include nucleic acids, peptides, receptors, and ligands.

In any of the variety of approaches for analyzing the biomolecules or detecting the presence of a target biomolecule, the identification molecule can, for example, comprise a capture molecule that retains a component of the specimen, for example in an array or in a layer. If this is done, the analysis can be completed by exposing the identification molecule to a detection molecule that associates with a combination of the capture molecule and the component of the sample, or associates with a region of the component different than the region that was recognized by the identification molecule. For example, the molecule of interest can be a protein, and the identification molecule can recognize a first domain of the protein, and the detection molecule recognizes a second domain of the protein.

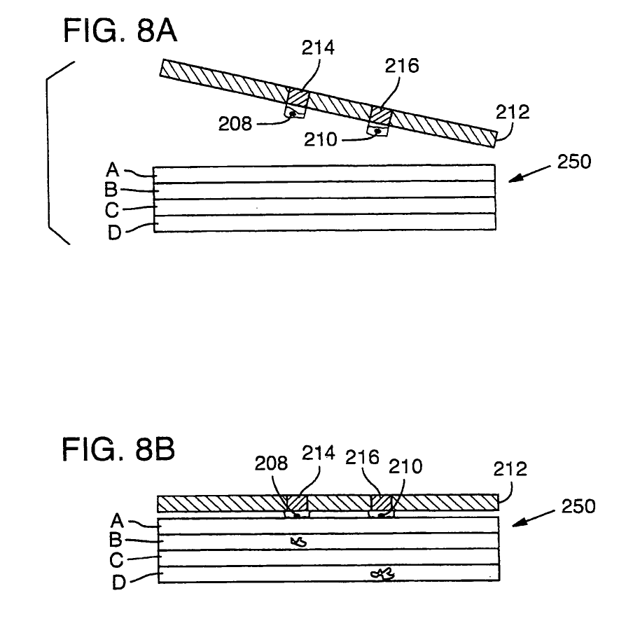

An example of this approach is shown in FIG. 8, in which the transfer member 212, discussed in connection with FIG. 7, is applied to the layered expression scanning device 250, which is this example is shown as having four layers A, B, C, and D. Transfer member 212 has adhered to it, at different preselected focal regions 214, 216, target regions 208, 210 (FIG. 8A). When transfer member is applied to the top of device 250, and biomolecules from target regions 208, 210 are moved through device 250, biomolecules that interact with different specific binding agents in different layers are retained by the binding agent in the respective layer. This is illustrated schematically in FIG. 8B, in which Protein 1 is shown to be retained in layer B at a position that corresponds to the location of target region 208 on the top layer of device 250, and Protein 2 is shown to be retained in layer D at a position that corresponds to the location of target region 210 on the top layer of device 250. As this example illustrates, the transfer member need not have focally activated regions through which the biomolecules of interest move, but instead the biomolecules can be obtained by focal adhesion to the transfer member, for subsequent application to an analysis substrate.

The capture molecule used in some embodiments of the layered expression scan has the ability to inhibit the transfer of at least some of one or more molecules of interest present in the specimen to a downstream region (such as a layer) of the analysis substrate. In some embodiments the method results in a pattern of capture that can be viewed as a plurality of two-dimensional patterns that, when stacked, forms a three-dimensional matrix. The two-dimensional patterns may, in specific embodiments, be spatially preserved, in that the patterns reflect the pattern of expression or presence of the molecule of interest within the cellular specimen, or the pattern of target regions adhered to the transfer member.

### EXAMPLE 9

### Specific Example of Laser Capture Microdissection Analysis

The tissue section is first microdissected according to the methods of Emmert-Buck et al., Science, 274: 998-1001, 1996, the contents of which are incorporated herein. However, instead of removing the captured cells from the LCM capture membrane, the LCM membrane is used directly for analysis in the substrate with the capture regions. The individual microdissected cells on the transfer membrane are then aligned with capture regions of an analysis substrate (such as nucleic acid probes in an array, or antibodies in microtiter wells). In effect, the LCM membrane serves a purpose similar to the transfer member in selectively transferring selected biomolecules from a biological sample to an analysis substrate.

The LCM membrane is capable of sustaining a flow of liquid into and through the analysis substrate, such as a nucleic acid array or layered expression scan. The thermoplastic capture membrane may be made of a material that is permeable to the solvent, typically water, that is used to carry molecules into the substrate for analysis. Ordinary ethylene vinyl acetate (EVA) capture films are not well suited for this purpose because of their hydrophobicity, but they may be altered to increase their hydrophilicity such that they become more permeable.

In specific examples, LCM capture films, such as films constructed from thermoplastics, for example, ethylene vinyl acetates (EVAs), polyurethanes (PU), polyvinyl acetates, ethylene, methyl acrylate (EMAC), polycarbonate (PC), ethylene-vinyl alcohol copolymers (EVOH), polypropylene (PP), and polystyrenes (PS) are used. These capture films are permeable to aqueous solutions or may be blended with other materials to improve their water permeability and their hydraulic conductivity of aqueous solutions, such as buffer solutions. PU and EVOH are examples of highly water permeable membranes. Fillers, such as salts, for example, non-hygroscopic salts, and water-soluble polymers, such a cellulose derivatives, may be added to the LCM capture film during their production to increase their water permeability. Methods of forming porous films by the addition of salts are disclosed in U.S. Pat. No. 3,844,865 to Elton et al., and in U.S. Pat. No. 3,870,593, also to Elton et al.

In some embodiments, a water-soluble polymer or fibers of such a polymer are added to the LCM capture film. Examples of suitable water-soluble polymers are disclosed in U.S. Pat. No. 4,618,648 to Martin. Specific examples of water-soluble polymers include cellulose ethers, acrylic acid-maleic anhydride copolymers, and carrageenan. Such polymers may dissolve by hydrolyzing in the solution used for transfer through the LES stack.

In yet other embodiments the thermoplastic film itself is water-soluble. Examples of water-soluble thermoplastic films are disclosed in Japanese Patent 62,070,075 to Hirotoshi et al.

The tissue section is laser capture microdissected according to the methods of Emmert-Buck et al., Science, 274: 998-1001, 1996, by fusing them to the thermoplasic film which covers the tissue section. The thermoplastic film is then removed from the tissue section and is applied to the substrate with the microdissected cells being positioned between the LCM capture membrane and the substrate. Buffer is added to the membrane to either dissolve the entire film or dissolve the water-soluble polymers or fibers of such polymers incorporated within the membrane. Water soluble polymers or fibers thereof dissolve to form microchannels, thereby permeabilizing the membrane to fluid flow. Transfer through the stack is then accomplished as described in Englert et al., Cancer Research, 60: 1526-1530, 2000, the contents of which are incorporated herein by reference, or in the manner already described in the present specification. In the case of thermoplastic films incorporating inorganic fillers, such as non-hygroscopic salts, the fillers themselves function as channels for fluid flow.

### EXAMPLE 10

### Layered Expression Scanning Analysis Substrate

Particular examples of materials appropriate for constructing a set of layers for layered expression scanning include nitrocellulose membranes, derivatized nitrocellulose membranes, high concentration agarose gels, low concentration agarose gels, high concentration polyacrylamide gels, a low concentration polyacrylamide gel, and membranes, such as porous membranes like nitrocellulose paper. Low concentration agarose is from about 0.1 to about 3%, while high concentration is above about 3%. Low concentration acrylamide is about 2% to about 20%, while high concentration is above about 20%.

Individual layers may also be composites of two or more membranes or gels. For example, thin polymer membranes, such as polar polymer membranes, for instance polyester membranes, may be combined with nitrocellulose membranes or agarose or polyacrylamide gels to form composite layers for layered expression scanning.

In a particular embodiment, the composite membrane is formed as follows. A thin (10 μm) polyester membrane is used as a backbone layer. The polyester membrane is then coated with a soluble polymer material, such as 2% agarose, to form an ultrathin (<1 μm) layer covering the polyester backbone. A capture molecule (e.g., an antibody or nucleic acid) is added to the polymer material prior to its addition to the polyester backbone. After the polymer is coated on the backbone, it forms a gel and irreversibly traps the capture molecule within the gel structure. The polyester backbone/polymer gel composite containing the capture molecule may then be used as a layered expression scanning capture membrane. Experiments have demonstrated that such composite membranes are highly efficient at meeting the criteria described above. A particular advantage of the composite membranes is that the polymer gel that is coated on the polyester backbone serves as a “contact substance” between each of the layers, thereby permitting efficient transfer of biomolecules with minimal loss of correspondence with the two-dimensional architecture in the sample.

### EXAMPLE 11

### Additional Examples of Alternative Materials and Methods

The nitrocellulose layers described herein can instead be substituted with porous polymer layers, for example made either from a hydrophilic polymer, or (for thin layers) from a mixture of hydrophilic and hydrophobic polymers, or an aqueous dissolvable crystal in hydrophobic polymers. Hydrophilic polymer fibers may be embedded in the hydrophobic polymer to help encourage unidirectional transport through the polymer layer, and inhibit lateral diffusion/transport of biomolecules as they travel through the layer.

Hydrophilic polymers that could replace nitrocellulose include (without limitation) neutral polymers such as methylcellulose, polyacrylamide, or polyvinyl alcohol; charged hydrophilic polymers such as polyacrylic acid, or most biopolymers (collagen, etc); hydropholic polymers such as silicone, polyethylene, polyethylene naphthalate, teflon, or polyvinyl acetate. Mixtures of hydrophilic polymer fibers (above) in hydrophobic polymers are examples of polymers that would enhance transverse porosity with bulk hydrophobicity.

Laser ablation of holes through the polymer layers is illustrated in this example. A PEN (polyethylene naphthalate) thin film (2 μm thick) is coated with a mixed hydrophobic polymer with hydrophilic polymer fibers (instead of placing an agarose gel layer adjacent the PEN film). The coating is surface treated with an electro beam, so the coating has a high affinity for the tissue section (e.g., surface charge). The tissue section is adhered on this charged surface. A Nitrogen laser (337 nm) or an excimer laser (353 nm) or a tripled Nd:YAG at (355 nm) is used to deliver a focal pulse of laser light to a spot on the PEN film (which strongly absorbs for all these lasers) on the spots overlying the tissue cells (targets) of interest in order to drill a hole through the hydrophobic-aqueous impermeable layer (PEN), but without damaging the tissue. Then a layered expression matrix (with multiple high affinity ligands—one per each layer—embedded in the hydrophilic fibers within an otherwise hydrophobic polymer) is placed on the PEN.

Since the bulk property of the transfer laminate is hydrophobic and that of the PEN film is hydrophobic, they can be made to have a high surface affinity which excludes water from all but the location where the pores were created or the affinity hydrophilic (layered) polymer fibers are. The laser ablation charges the PEN polymer channel surfaces so that they are hydrophilic.

Alternatively a GaAs laser diode at 807 nm (or similar wavelength matched to the specific IR absorbing dye) is used to ablate or melt (assuming a foamant is in the polymer film) a polymer film such as any of the above hydrophobic polymers containing the appropriate dye (for example vanadyl naphthalocyanine for a 807 nm laser). Hydrophobic polymer layers as thin as 200 nm have been made for this purpose with almost total absorption of 807 nm laser (99%).

In view of the many possible embodiments to which the principles disclosed herein may be applied, it should be recognized that the illustrated embodiments are only particular examples of the invention, and should not be taken as a limitation on the scope of the invention. Rather, the invention includes all that comes within the scope and spirit of the following claims.

### REFERENCES

W. Bonte, Acta histochem. 62: 68-77 (1978).

Z. Chen et al., Clin. Chem. 41:1273-82 (1995).

M. Emmert-Buck et al., Science 274: 998-1001 (1996).

M. Inczedy-Marcsek et al., Acta Histochem. Supp. 36: S377-94 (1988).

T. Grogan, Am. J. Clin. Pathol. 4(Supp. 1): S35-8 (1992).

Z. Huang et al., Anal Biochem 268:305-17 (1999).

L. Jin and R. Lloyd, J. Clin. Lab. Anal. 11:2-9 (1997).

J. Kononen et al., Nat. Med. 4: 844-847 (1998).

I. Lefkovits et al. (eds.), Immunology Methods Manual (1996).

J. Lindner et al., Naturwissenschaften 43: 201 (1956).

V. Neuhoff, Electrophoresis '79 (1980).

C. Saravis et al., J. Immun. Meth. 29:97-100 (1979).

U. Schumacher et al. (1990), Histochem. J. 22:433-438 (1990).

U. Schumacher and D. Trudrung, Anal. Biochem. 194: 256-58 (1991).

M. Schena et al., Science 270: 467-469 (1995).

K. Smith et al., Proc. Natl. Acad. Sci. 90: 2846-2850 (1993).

P. van der Sluis et al., Electrophoresis 9: 654-66 (1988).

L. Zhang et al., Science 276: 1268-1272 (1997).

S. Zucker et al., Clin, Exp. Metastasis 12:13-23 (1994).

We claim:

1. A method of analyzing a biological sample, comprising:
placing a transfer member adjacent the biological sample;
activating a discrete portion of the transfer member adjacent a target region of the biological sample, wherein activation of the discrete portion of the transfer member changes a physical characteristic of the transfer member to selectively increase permeability of the transfer member to biomolecules of the target region; and
moving biomolecules in the target region through the transfer member for subsequent analysis of the biomolecules.

2. The method of claim 1, wherein the target region is selected under a microscope.

3. The method of claim 1, wherein the target region is an area of biologically distinct cells.

4. The method of claim 1, wherein the activation comprises decreasing a density of the transfer member to increase movement of the biomolecules through the transfer member.

5. The method of claim 4, wherein decreasing the density comprises removing the discrete portion of the transfer member.

6. The method of claim 5, wherein the activation comprises increasing a solubility of the discrete portion to at least some of the biomolecules.

7. The method of claim 6, wherein increasing the solubility comprises increasing a solubility of one or more biomolecules of interest in the transfer member.

8. The method of claim 1, further comprising applying a recipient member to the transfer member, wherein at least some of the biomolecules that move through the transfer member are retained by the transfer member.

9. The method of claim 1, wherein moving biomolecules through the transfer member comprises moving the biomolecules in a liquid.

10. The method of claim 9, wherein the liquid is moved by capillary action.

11. The method of claim 10, further comprising placing an absorbent member adjacent the transfer member, in a position that draws the liquid through the biological sample and the transfer member.

12. The method of claim 1, wherein the biological sample is a tissue specimen.

13. The method of claim 1, wherein the subsequent analysis comprises interacting the biomolecules with a specific binding agent.

14. The method of claim 13, wherein the specific binding agent is an antibody or a nucleic acid.

15. The method of claim 13, wherein the specific binding agent comprises a plurality of specific binding agent elements arranged in an array on a substrate.

16. The method of claim 15, wherein the array is an array of arrays.

17. The method of claim 15, wherein the transfer member is activated between the biological sample and one or more elements of the array.

18. The method of claim 17, wherein the transfer member is activated between the biological sample and one or more arrays of the array of arrays.

19. The method of claim 13, wherein the specific binding agents are contained in contiguous layers of an analysis substrate.

20. The method of claim 19, wherein different specific binding agents are contained in different contiguous layers of the analysis substrate.

21. A method of analyzing a biological specimen for the presence of one or more target biomolecules, comprising:
contacting a biological sample with a selectively activatable transfer member which can be activated to selectively isolate a portion of the tissue sample;
identifying at least one portion of the tissue sample which is to be extracted;
selectively activating a region of the transfer member which corresponds to and is in contact with the at least one portion of the tissue sample so that the activated region of the transfer member selectively isolates the at least one portion of the tissue sample; and
applying the at least one portion of the tissue sample that has been isolated to a substrate comprising a plurality of identification molecules, and contacting biomolecules from the one or more cells with the plurality of identification molecules to determine if the one or more target biomolecules is present.

22. The method of claim 21, wherein selectively activating a region of the transfer member comprises:
(a) adhering the region of the transfer member, and isolating comprises separating the transfer member from the tissue sample while maintaining adhesion between the activated region of the transfer member so that the at least one portion of the tissue sample is extracted from a remaining portion of the tissue sample; or
(b) removing the region to expose the at least one portion of the tissue sample.

23. The method of claim 21, wherein the substrate comprises different analysis stations, each station including a plurality of identification molecules.

24. The method of claim 23, wherein the different analysis stations comprise different nucleic acid molecules.

25. The method of claim 24, wherein the different analysis stations comprises separate nucleic acid arrays on a surface of the substrate.

26. The method of claim 25, wherein the separate nucleic acid arrays comprise different nucleic acid arrays.

27. The method of claim 23, wherein the plurality of different identification molecules comprise different antibodies.

28. The method of claim 21, wherein the substrate comprises a plurality of different layers, with different identification molecules in at least some of the different layers.

29. The method of claim 23, wherein the substrate comprises a plurality of chambers with at least one nucleic acid array, wherein each of the plurality of chambers is no greater than 300 μm wide, and the one or more cells is no more than 100 cells.

30. A product comprising the transfer member and substrate of claim 21.

31. A method of analyzing a biological specimen, comprising:
placing the biological specimen on a substrate with one or more different capture regions, wherein the one or more different capture regions of the substrate contain different identification molecules that interact with different biological molecules; and
transferring components of one or more targeted locations of the biological specimen through the capture regions under conditions that allow the components to interact with the different identification molecules in the different regions of the substrate to produce a pattern that is informative about the identification of the biological molecule.

32. The method of claim 31, wherein the different regions of the substrate are different layers.

33. The method of claim 31, wherein the biological specimen is a cellular specimen.

34. The method of claim 31, wherein components of targeted locations of the biological specimen are transferred by placing the biological specimen on a transfer member, and selectively altering the transfer member to transfer targeted locations of the biological specimen through the transfer member into the substrate.

35. The method of claim 34, wherein altering the transfer member comprises fusing the targeted locations from the biological specimen to the transfer member, then exposing the fused targeted locations to a surface of the substrate.

36. The method of claim 35, wherein the targeted locations are fused to the transfer member by a laser.

37. The method of claim 36, wherein the targeted locations are fused to the transfer member by laser capture microdissection.

38. The method of claim 34, wherein altering the transfer member comprises removing portions of the transfer member at the targeted locations.

39. The method of claim 38, wherein removing portions of the transfer member comprises ablating the portions of the transfer member with heat or radiant energy.

40. The method of claim 39, wherein the radiant energy is a laser beam.

41. The method of claim 34, wherein altering the transfer member comprises locally changing a permeability of the transfer member in the targeted locations.

42. The method of claim 32, wherein the layers of the substrate are contiguous, and components of the specimen at the targeted locations are transferred through the different layers of the substrate by capillary action of the substrate.

43. The method of claim 32, wherein the layers of the substrate comprises contiguous porous layers that exert capillary pressure on the specimen.

44. The method of claim 31, wherein the components of the specimen at the targeted locations are transferred through the different layers of the substrate by electrophoresis.

45. The method of claim 32, wherein the biological specimen is a cellular specimen, and the layers of the substrate maintain a cellular architecture of the specimen as the specimen is transferred through the layers of the substrate.

46. The method of claim 45, further comprising correlating interaction between different identification molecules and the components of the cellular specimens, with a cellular architecture of the specimen.

47. The method of claim 32, further comprising placing multiple different discrete cellular specimens on a surface of the substrate, wherein a correspondence is maintained between the multiple discrete cellular specimens and particular transferred components.

48. The method of claim 37 wherein at least 20 different cellular specimens are placed on the surface of the substrate.

49. The method of claim 33, wherein the cellular specimen is a section of a tissue specimen.

50. The method of claim 49, wherein the cellular specimen is a section of a tumor.

51. The method of claim 32, further comprising correlating a pattern of interactions of different identification molecules in the different layers of the substrate with a specimen having a known identity.

52. The method of claim 32, wherein there are at least 10 layers of the substrate.

53. The method of claim 52, wherein there are at least 20 layers of the substrate.

54. The method of claim 53, wherein there are at least 100 layers of the substrate.

55. The method of claim 54, wherein there are at least 1000 layers of the substrate.

56. The method of claim 32, wherein the layers of the substrate have a thickness of at least about 25 μm.

57. The method of claim 33, wherein the identification molecules are antibodies that interact with the components of the cellular specimen.

58. The method of claim 33, wherein the identification molecules interact with different cellular regions of the cellular specimen, and interaction of the identification molecules is correlated with a region of the cellular specimen.

59. The method of claim 33, comprising identifying the component of the specimen by determining which identification molecule the component interacts with.

60. The method of claim 32, wherein the layers of the substrate comprise electrically conductive gel layers.

61. The method of claim 60, wherein the gel layers are separable, and are separated after transferring the components of the cellular specimen, for individualized identification of the components of the specimen retained in each separated layer.

62. The method of claim 31 wherein the each layer of the substrate is water permeable.

63. The method of claim 32 wherein the identification molecules are molecules selected from the group consisting of antibodies, nucleic acids, peptides, receptors, and ligands.

64. The method of claim 32 wherein the identification molecules comprise capture molecules which retain a component of the specimen in the layer, the method further comprising exposing the identification molecule to a detection molecule that associates with a combination of the capture molecule and the component of the sample, or associates with a region of the component different than a region that is recognized by the identification molecule.

65. The method of claim 64, wherein the component is a protein, the identification molecule recognizes a first domain of the protein, and the detection molecule recognizes the different region of the protein.

66. The method of claim 64, wherein the detection molecule is selected from the group consisting of antibodies, nucleic acids, peptides, receptors, ligands and stains.

67. The method of claim 33, wherein the identification molecules capture the components of the cellular biological specimen in relative abundance to a quantity of the components in the targeted locations of the cellular specimen, and provide a quantitative indication of the relative abundance of the components in the cellular specimen.

68. The method of claim 33, wherein the cellular specimen is selected from the group consisting of a tissue section, cultured cells, and a cytology sample.

69. The method of claim 31, wherein the transferred components that interact with the different identification molecules comprise intact proteins or intact nucleic acid molecules that have not been subjected to proteolytic or nucleolytic reactions prior to transfer through the different layers of the substrate.

70. The method of claim 31, wherein transferring components of one or more targeted locations of the biological specimen through the substrate produces a three dimensional matrix, wherein a surface of the substrate on which the biological specimen is placed provides a two dimensional matrix, and a third dimension is provided by transfer of components of the biological specimen through the different layers, wherein there is an identifiable correspondence between a position of the component of the specimen in the two dimensional matrix and a position of the transferred components in the three dimensional matrix.

71. The substrate with the three dimensional matrix of claim 70.

72. A method of analyzing a cellular specimen, comprising:
providing a substrate comprising a plurality of different layers having contiguous faces, each layer including a corresponding capture molecule capable of interacting with and capturing a component of the cellular specimen;
applying the cellular specimen to a transfer member, and selectively altering the transfer member at targeted locations, and transferring components of the cellular specimen at the targeted locations through the altered locations, into the substrate, and through the contiguous faces of the different layers of the substrate;
reacting the components of the cellular specimen with the capture molecules; and
correlating a pattern of capture in the different layers with information about the cellular specimen.

73. The method of claim 72, wherein the capture molecule captures the component in an amount that corresponds to a quantity of the component in the cellular specimen.

74. The method of claim 73, wherein the components comprise one or more of proteins or nucleic acids that have not been subjected to a proteolytic or nucleolytic processing step.

75. The method of claim 74, wherein transferring the components of the cellular specimen comprises introducing an electrical current through the transfer member and contiguous faces of the substrate, so that the current flows transverse to the plurality of different layers, and the plurality of different layers comprises a plurality of contiguous electrically conductive gels through which the electrical current is conducted.

76. The method of claim 74, wherein transferring the components of the specimen comprises transferring the components by capillary action through the transfer member and the substrate.

77. The method of claim 76, wherein the plurality of different layers comprise contiguous nitrocellulose layers that exert capillary pressure on the cellular specimen.
